(19) United States
(12) Patent Application Publication
(10) Pub. No.: US 2003/0219491 A1
(43) Pub. Date: Nov. 27, 2003

(54) Compositions for inhibiting tumor growth and methods thereof

(76) Inventors: Thomas Chang, St. Lambert (CA); Binglan Yu, Montreal (CA)

Correspondence Address: WAYNE A. KEOWN, SUITE 1200, 500 WEST CUMMINGS PARK, WOBURN, MA, 01801, US

(21) Appl. No.: 10/391,340

(22) Filed: Mar. 18, 2003

Related U.S. Application Data
(60) Provisional application No. 60/364,584, filed on Mar. 18, 2002.

(51) Int. Cl. A61K 38/48; A61K 38/21; A61K 38/20; A61K 9/16

(52) U.S. Cl. 424/490; 424/85.2; 424/85.5; 424/85.6; 424/85.7; 514/6; 424/94.63

(57) ABSTRACT

The present invention relates to a composition for inhibiting tumor growth, which comprises a tumor growth inhibiting enzymatic moiety in association with at least one protective carrier. The present invention further relates to the methods of inhibiting tumor growth using this composition.

This U.S. application claims priority on the U.S. application Ser. No: 60/364,581 filed on Mar. 18, 2002.

### BACKGROUND OF THE INVENTION

a) Field of the Invention

This invention relates to a new composition for inhibiting tumor growth and methods of treatment thereof.

(b) Description of Prior Art

Melanoma, a fatal skin cancer, now represents the fifth most common type of cancer in North America. The incidence rate of melanoma has risen dramatically in the last century in all countries with a white-skinned population, doubling every 10 years in many countries, and is now approximately 10 per 100,000 per annum in Europe, giving an approximate lifetime risk of 1 in 200 (Katsambas, A. & Nicolaidou, E. Cutaneous malignant melanoma and sun exposure. Arch. Dermatol. 132, 444-450 (1996)). It is also reported that at least 20% of people diagnosed with melanoma will experience advanced disease and die within 5 years of diagnosis (Beahers, O. H. , Henson, D. E. , Hutter, R. V. P. & Kennedy, B. J. Malignant melanoma of the skin (excluding eyelid). American Joint Committee on Cancer Manual for staging of cancer 4^{th} ed. Philadelphia: JB Lippincoft. 143-148 (1992)). At present there is no optimal treatment for this cancer. Adjuvant therapy with varying clinical results includes immunotherapy, such as interferon α-2b (Caraceni, A. et al. Neurotoxicity of interferon-α in melanoma therapy. Cancer 83, 482489 (1998)) levamisole, vaccines, chemotherapy, autologous bone marrow transplantation, biochemotherapy and chemoimmunotherapy (Johnson, T. M. , Yahanda, A. M. , Chang, A. E. , Fader, D. J. & Sondak, V. K. Advances in melanoma therapy. J. Am. Acad. Dermatol. 38, 731-741 (1998)). Thus, there is at present no conclusive method for the treatment of melanoma, a fatal skin cancer.

One unique characteristic is that tyrosine requirement for malignant melanomas is much higher than for normal cells since tyrosine is needed for both protein and melanin synthesis. Tyrosine is a semi-essential amino acid, derived from the liberation of tyrosine from hydrolysis of dietary or tissue protein. Dietary approach to lower tyrosine was not successful in human because it results in malnutrition in the severely sick cancer patients, its unpalatable nature also make compliance in human difficult, furthermore, it took a long time to lower systemic tyrosine (Johnson, T. M., Yahanda, A. M., Chang, A. E., Fader, D. J. & Sondak, V. K. Advances in melanoma therapy. J. Am. Acad. Dermatol. 38, 731-741 (1998)). The injection in human of an enzyme, tyrosinase, by itself was also not practical (Johnson, T. M., Yahanda, A. M., Chang, A. E., Fader, D. J. & Sondak, V. K. Advances in melanoma therapy. J. Am. Acad. Dermatol. 38, 731-741 (1998)) because the short half-life required repeated injection resulting in immunological problems from the bare enzyme.

It would be highly desirable to be provided with a new composition for inhibiting tumor growth.

### SUMMARY OF THE INVENTION

In accordance with the present invention there is provided a new composition and method for inhibiting tumor growth.

In accordance with the present invention, there is provided a composition for inhibiting tumor growth, which comprises a tumor growth inhibiting enzymatic moiety in association with at least one protective carrier.

The composition in accordance with a preferred embodiment of the present invention, wherein the carrier is an oral encapsulation carrier to protect encapsulated enzymatic moiety from digestive enzyme degradation.

The composition in accordance with a preferred embodiment of the present invention, wherein the encapsulation carrier is a nanocapsule.

The composition in accordance with a preferred embodiment of the present invention, wherein the enzymatic moiety is selected from the group consisting of tyrosinase, asparaginase and glutaminase.

The composition in accordance with a preferred embodiment of the present invention, further comprising an oxygen binding molecule.

The composition in accordance with a preferred embodiment of the present invention, wherein the carrier is a molecule biologically active and protective that covalently bond to the enzymatic moiety.

The composition in accordance with a preferred embodiment of the present invention, wherein the molecule increase half-life of the enzymatic moiety.

The composition in accordance with a preferred embodiment of the present invention, wherein the molecule is an oxygen binding molecule.

The composition in accordance with a preferred embodiment of the present invention, wherein the molecule is hemoglobin or synthetic hemoglobin.

The composition in accordance with a preferred embodiment of the present invention, wherein the molecule is albumin or antineoplastic molecule.

The composition in accordance with a preferred embodiment of the present invention, wherein the antineoplastic molecule is selected from the group consisting of interleukin, interferon (α, interferon β and interferon γ.

In accordance with the present invention, there is provided a method for inhibiting tumor growth, the method comprising the step of administering the composition the present invention to a patient.

The method in accordance with a preferred embodiment of the present invention, wherein the enzymatic moiety is tyrosinase (or enzymes with tyrosinase-like activity for degrading tyrosinase) and the tumor is a skin cancer tumor.

The method in accordance with a preferred embodiment of the present invention, wherein the skin cancer is melanoma.

The method in accordance with a preferred embodiment of the present invention, wherein the enzymatic moiety is selected from the group consisting of asparaginase and glutaminase.

The method in accordance with a preferred embodiment of the present invention, wherein the tumor is selected from the group consisting of asparinase and gluataminase could be used in leukemia or lymphomas that depends on asparagines or glutamine for growing.

For the purpose of the present invention the following terms are defined below.

The term “oxygen carrying molecule” is intended to mean hemoglobin, synthetic hemoglobin, synthetic hemes or any other oxygen carrying molecules.

The term “protective carrier” is intended to mean a carrier that prevents degradation of the enzymatic moiety and therefore increase its half-life.

The term “molecule with enzymatic activity” is intended to mean synthetic enzyme, enzyme or functional fragment thereof.

The term “enzyme” is intended to mean tyrosinase, asparaginase, glutaminase or any other enzymes that can deplete any specific amino acid required for growth by any tumours.

All references herein referred to are incorporated by reference.

### BRIEF DESCRIPTION OF THE DRAWINGS

FIG. 1 illustrates the lowering of tyrosine by tyrosinase before and after crosslinking;

FIG. 2 illustrates the typical elution profile of PolyHb or PolyHb-tyrosinase;

FIG. 3 illustrates the activity of tyrosinase at 37° C. in vitro;

FIG. 4 illustrates the oxygen dissociation curve of pure bovine hemoglobin in free form and when it is crosslinked with tyrosinase to form the polyhemoglobin-tyrosinase complex;

FIGS. 5A-D illustrate the activity of polyhemoglobin(polyHb)-tyrosinase at different time intervals: A-3.5 hr, B-24 hr, C-30 hr and D-48 hr ;

FIG. 6 illustrates the Tyrosine concentration in rat's plasma after injection of PolyHb-tyrosinase;

FIG. 7 illustrates in vitro growth curves of B16F10 cell lines when incubated with polyhemoglobin-tyrosinase;

FIG. 8 illustrates enzyme activity in artificial cells at different volumes concentrations;

FIG. 9A illustrates the activity of free tyrosinase (1020 U/3 ml) in different concentration of tyrosine;

FIG. 9B illustrates a double-reciprocal plot of free tyrosinase (1020 U/3 ml);

FIG. 10A illustrates the activity of encapsulated tyrosinase (1020 U/3 ml);

FIG. 10B illustrates a double-reciprocal plot of encapsulated tyrosinase (1020 U/3 ml);

FIG. 11 illustrates the activity of free tyrosinase and encapsulated tyrosinase at different pH;

FIG. 12 illustrates encapsulated tyrosinase activity after incubated at 37° C. for 1 hour at different pH;

FIG. 13 illustrates storage stability of free and encapsulated tyrosinase at 4° C.;

FIG. 14 illustrates storage stability of free and encapsulated tyrosinase at 37° C.;

FIGS. 15A-C illustrates tyrosine concentration in rat's intestine juice after incubated with encapsulated tyrosinase (450 U/200 ul; 670 U/300 ul and 900 U/400 ul) in vitro;

FIG. 16 illustrates the body weight (g) of rats for control group fed with encapsulated Hb and test group fed with encapsulated Hb+tyrosinase twice a day for 21 days;

FIG. 17 illustrates the tyrosine level in test group in rat's plasma expressed as percentage of those in control group;

FIG. 18 illustrates the body weight (g) of rats for 22 days experiments;

FIG. 19 illustrates the tyrosine level in test group in rat's plasma expressed as percentage of those in control group; and

FIG. 20 illustrates the tyrosine concentration in rat's plasma (%).

### DETAILED DESCRIPTION OF THE INVENTION

In accordance with the present invention, there is provided compositions and methods for inhibiting tumor growth.

It has been found that a novel polyhemoglobin-tyrosinase preparation can rapidly lower the body tyrosine level after one intravenous injection. In this form, the enzyme is covered by hemoglobin molecules and therefore has less immunological properties. Furthermore, polyhemoglobin is an oxygen carrier and being a solution, it can more easily reach the narrower capillaries of the melanoma cancer cells than red blood cells and can therefore bring more oxygen. The presence of high concentration of oxygen is important in radiotherapy for cancer cells. In vitro studies show that this novel polyhemoglobin-tyrosinase preparation inhibits the growth of melanoma cells in culture.

It also has been shown in accordance with the present invention that daily oral administration of encapsulated tyrosinase by itself for about 3-5 days, can lower the body tyrosine. The oral administration approach has the advantage that no intravenous injections are needed but takes a bit longer to lower the tyrosine level. A proposed encapsulation process is described in U.S. Pat. No. 5,084,350, which is incorporated by reference herein.

One intravenously injection of polyhemoglobin-tyrosinase followed by 3 times a day oral administration of encapsulated tyrosinase can lower the body tyrosine and maintain this low level as long as the oral administration is continued.

### Polyhemoglobin-tyrosinase Preparation

Materials

L-tyrosine (98% TLC), hemoglobin from bovine (lyophilized powder), hemoglobin assay kit, tyrosinase from mushroom (3400 U/mg manufacturer's stated activity) were purchased from Sigma-Aldrich (Oakville, Canada). Collodion was obtained from Fisher Scientific (Nepean, Canada). Glutaraldehyde (25%) was obtained from Polyscienes (Warrington, Pa.). Purified bovine hemoglobin was purchased from Biopure Corporation (Boston, Mass.). All other reagents were of analytical grade.

Preparation of the novel PolyHb-Tyrosinase composition Reaction mixtures were prepared containing hemoglobin 10 gd/l, tyrosinase (6000 U/ml) in 0.1 M potassium phosphate buffer, pH 7.6. In PolyHb mixtures, an equivalent volume of buffer replaced enzyme condition. Prior to the start of crosslinking, 1.3 M lysine was added at a molar ratio of 7:1 lysine/hemoglobin. Crosslinking reaction was started with the addition of glutaraldehyde at molar ratio of 16:1 glutaraldehyde/hemoglobin. Glutaraldehyde was added in four equal aliquots over a period of 15 minutes. After 24 h under aerobic conditions with constant stirring, reaction was stopped with 2.0 M lysine at a molar ratio of 200:1 lysine/hemoglobin. Solutions were analyzed in physiological saline solution overnight and pass through sterile 0.45 μM filter. Aliquots (maximum 500 μl) of the 16:1 crosslinked preparation were concentrated using 100 kDa microconcentrators (Amicon, Beverly, Mass.). Samples were centrifuged at 2500 for 55 min at 23° C. Then, retentate was collected. Hemoglobin concentration was determined by cyanomethemoglobin at 540 nm. Final retentates were diluted to desired concentration at 7 g/dl and 4° C. fridge for later injection

### Analysis of Hemoglobin-tyrosinase and Polyhemoglobin-tyrosinase Activity

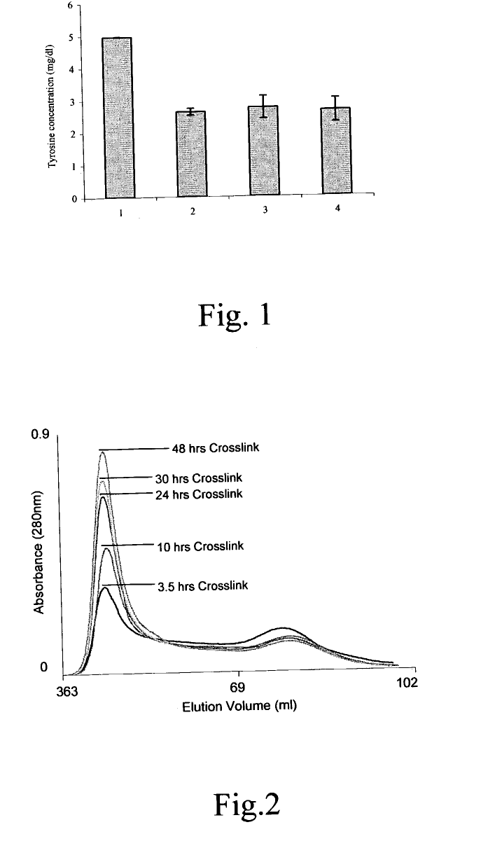

This is to study the effects of crosslinking on the tyrosinase enzyme activity. In the study of the effect of glutaraldehyde ratio (one of the crucial reagents for crosslinking ) on tyrosinase activity, tyrosinase activity was tested with the addition of glutaraldehyde at different molar ratio of 8:1 and 16:1 glutaraldehyde/hemoglobin. Hemoglobin was considered with tyrosinase activity without addition of glutaraldehyde as 100% original activity. In table 1, after crosslinking, 99% of enzyme activity remained in PolyHb-Tyrosinase at gluataraldehyde molar ratio of 8:1. For PolyHb-tyrosinase at glutaraldehyde molar ratio of 16:1, 95% activity was obtained. Therefore, no significant difference in enzyme activity between these two groups. Then, hemoglobin was crosslinked with tyrosinase from 3 h to 24 h, and took samples for enzyme activity analysis every 2 h. Results showed that no significant difference in tyrosinase activity among these periods. It indicated that up to 24 h crosslinking time did not decrease enzyme activity significantly. In FIG. 1, the enzyme activity was measured before and after crosslinking. PolyHb served as control, then enzyme activity was tested before and after crosslinking or non-crosslinking which buffer instead of glutaraldehyde solution. From the results obtained, there was no significant change in enzyme activity before or after crosslinking. This further confirmed that crosslinking reaction does not affect enzyme activity significantly.

TABLE I
Tyrosinase activity after crosslinking to form PolyHb-Tyrosinase

| Samples | retained | % Tyrosinase |
|---|---|---|
| Hemoglobin + Tyrosinase | | 100 |
| PolyHb-Tyrosinase at | 8:1 | 99 |
| Glut:Hb | | |
| PolyHb-Tyrosinase at | 16:1 | 95 |
| Glut:Hb | | |

Molecular Weight Distribution of PolyHb and PolyHb-Tyrosinase

To determine the degree of polymerization, samples were analyzed by gel filtration chromatography. At different reaction time, i.e. 3.5 h, 10 h, 24 h, 30 h, 48 h, run sample on a Sephadex G-200 1.6 cm×70 cm column. From the results obtained, the molecular weight distributions were the same for PolyHb and PolyHb-Tyrosinase (FIG. 2). In FIG. 2, typical elution profile of PolyHb or PolyHb-tyrosinase (1 ml sample) run on a Sephadex G-200 1.6 cm×70 cm column, VT=102 ml, equilibrated with 0.1 M Tris-HCI, pH 7.5, and eluted at 12 ml/hr.

Results showed that two main molecular weight distributions were found at 600 kDa and 60 kDa. The longer time the sample was crosslinked, the higher peek was found at or over 600 kDa. The ratio of hemoglobin to tyrosinase was 1:0.02. The added tyrosinase therefore is not expected to significantly change the molecular weight distribution after being crosslinked with hemoglobin. Table 2 shows the percentage of molecular weight distribution.

TABLE 2
Percentage of area under molecular distribution profiles

| | | Percentage of Molecular Weight | | |
|---|---|---|---|---|
| | | Distribution (KD) | | |
| Crosslinking | | Greater than | Between 61 KD | Less than |
| Time (hours) | Group | 600 KD | and 600 KD | 60 KD |
| 3.5 hours | PolyHb | 37% | 33% | 30% |
| | PolyHb- | 37% | 33% | 30% |
| | Tyrosinase | | | |
| 10 hours | PolyHb | 55% | 23% | 22% |
| | PolyHb- | 55% | 23% | 22% |
| | Tyrosinase | | | |
| 24 hours | PolyHb | 67% | 20% | 13% |
| | PolyHb- | 67% | 20% | 13% |
| | Tyrosinase | | | |
| 30 hours | PolyHb | 69% | 19% | 12% |
| | PolyHb- | 69% | 19% | 12% |
| | Tyrosinase | | | |
| 48 hours | PolyHb | 71% | 18% | 11% |
| | PolyHb- | 71% | 18% | 11% |
| | Tyrosinase | | | |

The stability of PolyHb-tyrosinase at 37° C.

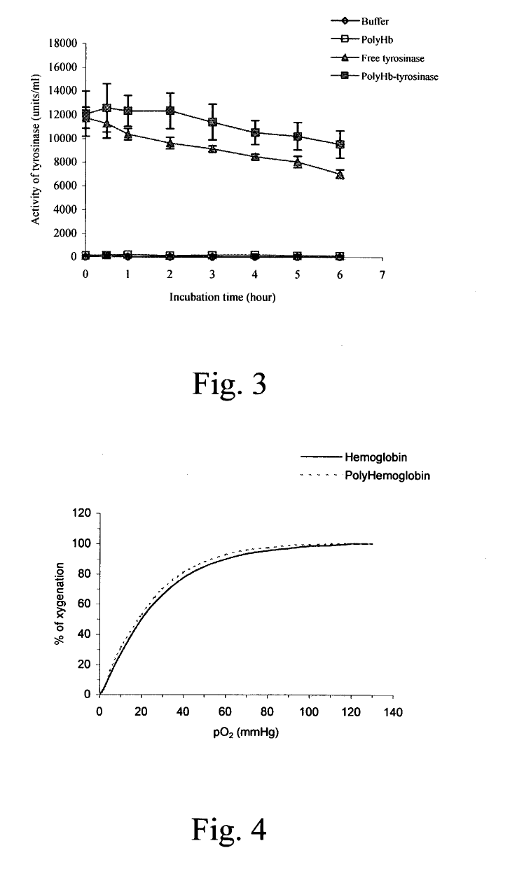

PolyHb-tyrosinase was incubated, free tyrosinase solution, buffer, PolyHb at 37° C. up to 6 hrs (FIG. 3). Results showed that enzyme activity in free tyrosinase solution decreased faster than PolyHb-tyrosinase at 37° C. At six hrs, 79% activity remained in PolyHb-tyrosinase compared to the activity at time 0. On the other hand, 60% activity was found in free tyrosinase solution after 6 h incubation.

### Oxygen Affinity of Hemoglobin and PolyHb

This is to study the effect of crosslinking tyrosinase to hemoglobin on the oxygen carrying and release characteristics of hemoglobin. Hemoglobin solution served as control group to compare with the novel polyhemoglobin-tyrosinase preparation. There was no significant difference in the oxygen release characteristics between the novel polyhemoglobin-tyrosinase preparation and free hemoglobin solution (FIG. 4). In FIG. 4, oxygen dissociation curve of pure bovine hemoglobin in the free form and in the crosslinking form (Crosslink time 24 h). There is no significant difference in P₅₀ between the two groups. This shows that the novel polyhemoglobin-tyrosinase preparation retains the ability to carry and release oxygen.

### The molecular Distribution of Tyrosinase Activity of PolyHb-tyrosinase Prepared by Different Degrees of Crosslinking

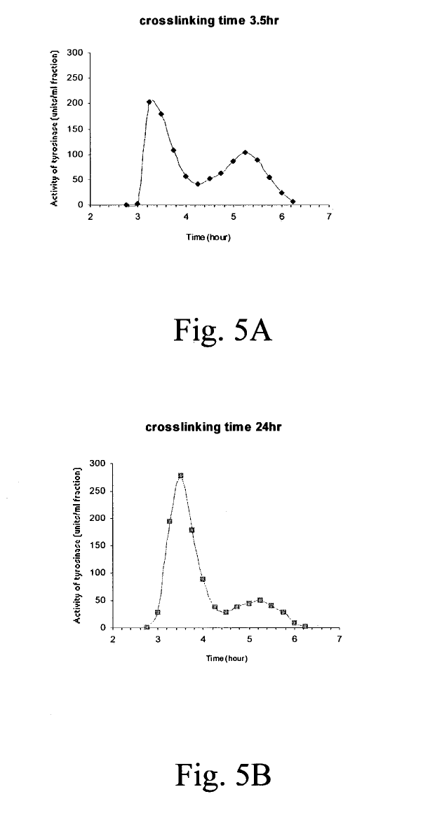

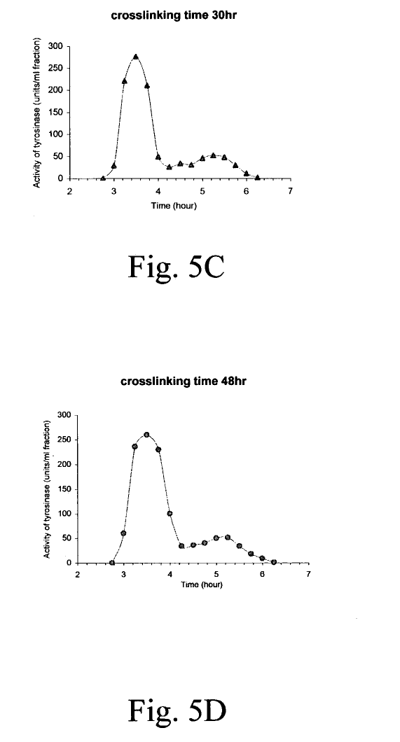

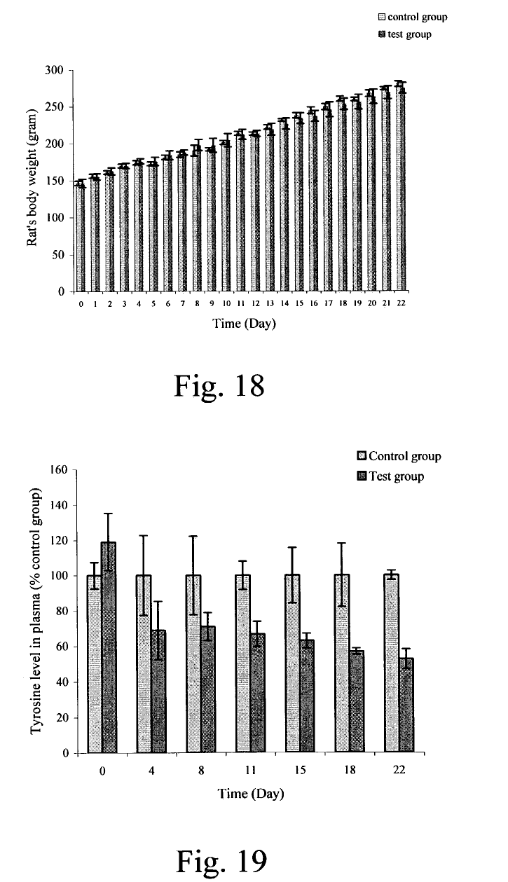

This is to study the molecular weight location of the tyrosinase in the polyhemoglobin-tyrosinase preparation. Hemoglobin was crosslinked with tyrosinase at 3.5 h, 24 h, 30 h and 48 h (FIGS. 5A-D). Then, took 1 ml PolyHb-Tyrosinase sample at different time intervals, run through Sephadex G-200 1.6 cm×70 cm column, equilibrated with 0.1M Tris HCI, and eluted at 12 ml/hr. In FIG. 18, the results obtained indicated that the longer time the sample was crosslinked, the higher enzyme activity was found at the molecular distribution of the polyhemoglobin fraction of 600 kDa. With the lowest crosslinking time of 3.5 hours, much of the tyrosinase are not crosslinked to the polyhemoglobin. With 24 hours or more crosslinking, most of the tyrosinase molecules were cross-linked to the polyhemoglobin fraction of 600 kDa.

### Animal Studies for Intravenous Injection

Fasted male Sprague-Dawley rats (245-260 g) were anaesthetized with intraperitoneal injection of pentobarbital (Somnotol, 65 mg/kg). Polyethylene cannulae were inserted and secured distal to the superficial epigastric branches in the femoral veins (PE-10, PE-50 Clay Adams). Take blood sample from each group at the beginning, then inject different samples through femoral vein. Connected femoral artery with vein and let blood circulate for a few seconds, then take blood samples from femoral artery at different time intervals.

### Determination of Tyrosine in Rat's Plasma

Tyrosine concentration in plasma was analyzed by fluorometric method using Perkin Elmer Luminescence Spectrometer LS50B .

### Statistical Analysis

The differences of tyrosine concentration in rat's plasma between two groups (control group and test group) at the same time point were determined by using Student's t-test within ANOVA and considered significant at P<0.05 35.

### Intravenous Injection Study of PolyHb-tyrosinase in Rats

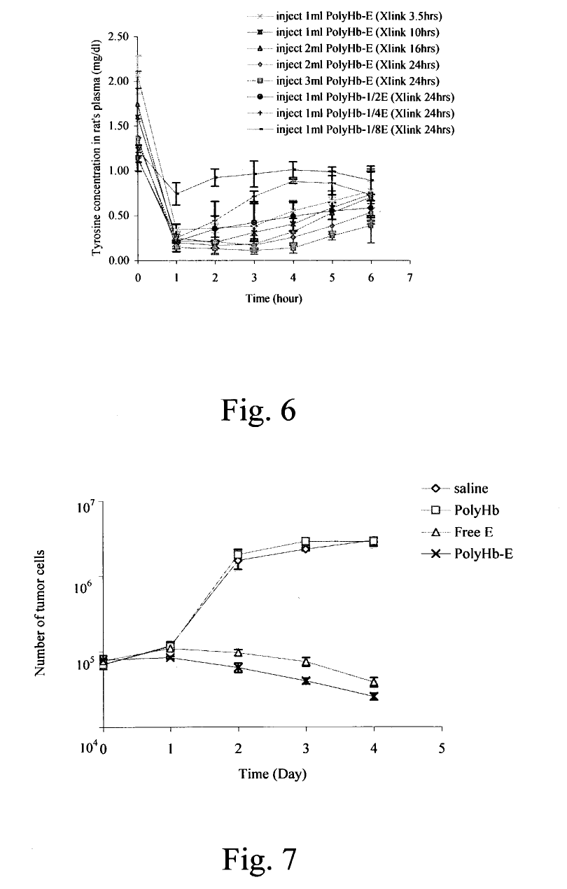

To investigate whether intravenous injection can lower tyrosine concentration in rats, polyhemoglobin-tyrosinase was injected at different hours and at different doses. Tyrosine level was rapidly decreased at the first hour after injection for all doses. However, the lowered levels were maintained more readily with the higher doses (FIG. 6).

### Studies of Melanoma Cell Culture

Tumor Cells and Culture Conditions B16-F10 murine melanoma cells were obtained from American Type Tissue Collection, Manassas Va. The tumor cells were routinely cultured in DMEM (Life Technologies, Invitrogen Canada) supplemented with 10% heat-inactivated FBS, sodium pyruvate, nonessential amino acids, 2-fold vitamin solution, L-glutamine, 100 lU/ml penicillin, and 100 lU/ml streptomycin at 37° C. in a humidified atmosphere of 5% CO2. For passage, cells were detached with 0.05% Trypsin-EDTA and transferred to fresh medium every 3 days. The cells were used in vitro between passage 5 and passage 10. For experiment, melanoma cells were cultured in complete DMEM until they became 30-40% confluent. Then, appropriate aliquots of different samples (0.57 ml sample per 10 ml medium) were added to the medium. The cell viability was followed up to 4 days thereafter.

In Vitro Cell Growth Assays

Tumor cells were routinely monitored by phase microscopy. Cell counts were obtained daily with a hemacytometer. Cell viability was determined by trypan blue exclusion.

To study whether polyhemoglobin-tyrosinase can inhibit the growth of melanoma cells, B16F10melanoma cells were cultured in DMEM with adding appropriate aliquots of saline solution, PolyHb solution, PolyHb-tyrosinase solution and free tyrosinase solution respectively. Cell growth was determined by trypan blue exclusion every day. Cells were counted at day 0 before the medium was added samples and on each day following. From the results obtained, after day 1, melanoma cells in saline solution and PolyHb solution were growing up. On the other hand, the cell growth in PolyHb-tyrosinase solution and free tyrosinase solution was decreasing because tyrosinase inhibits the growth of melanoma cells (FIG. 7).

### Encapsulated Tyrosinase for Oral Administration

Preparation of Microencapsules containing Tyrosinase for Oral Administration

One gram hemoglobin and 200 mg This was dissolved in 10 ml double distilled deionized water. Stir with a metal rod until everything is dissolved. Gravity filter the solution through a Waterman #42 filter into a Erlenmeyer flask. Take 2.5 ml of this 10 g/dl hemoglobin solution and was encapsulated within spherical, ultrathin, cellulose nitrate membrane. Without tyrosinase loaded microcapsules were administrated orally to control group. For tyrosinase loaded microencapsules, 1.5 mg of 3400 U/mg tyrosinase was dissolved in 2.5 ml 10% hemoglobin solution, then followed the methods described above to immobilize tyrosinase in collodion membrane microcapsules. Microcapsules prepared as a 50% suspension for later feeding. Tyrosinase loaded microencapsules were administered orally to test group. All microcapsules were prepared daily and stored in 1% v/v Tween 20 solution at 4° C. until use.

### Analysis of Free Tyrosinase and Encapsulated Tyrosinase Activity

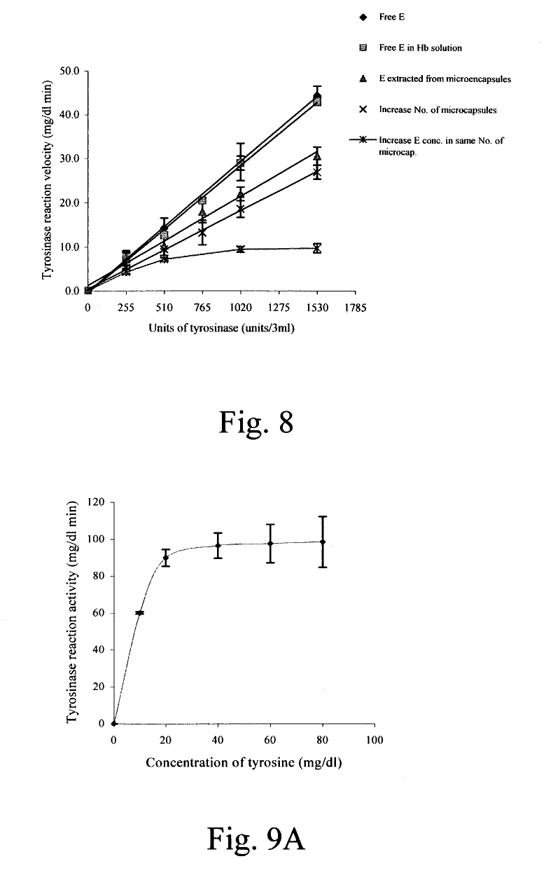

This is to optimize the preparation of encapsulated tyrosinase for use in oral administration. FIG. 8 shows the activity of free and encapsulated tyrosinase.

For free enzyme, increase in tyrosinase concentration resulted in increasing in reaction velocity. There is no significant difference in activity between free enzyme and free enzyme in hemoglobin solution. Thus, the interference due to the presence of hemoglobin component in the reaction mixture can be ruled out. For encapsulated enzyme, the reaction activity is increased when the concentration of tyrosinase inside the microcapsules is kept constant, but the volume of microcapsules is increased. However, the activity obtained is lower than enzyme in free solution. To study the reason for this, the membrane of encapsules was broken, then the activity of enzyme was tested. The results show that the activity of enzymes released from the encapsules is only very slightly higher than that of the intact microcapsules, showing that the decrease in activity is due to inactivity of some enzyme during the preparation procedure of microcapsules or some of enzyme entrapped inside the membranes rather than permeability restrictions. On the other hand, study was carried out when the tyrosinase concentration was increased stepwise but the volume of microcapsules was kept constant, the reaction activity is not increased corresponding to the concentration.

### Studies in Vmax and Km

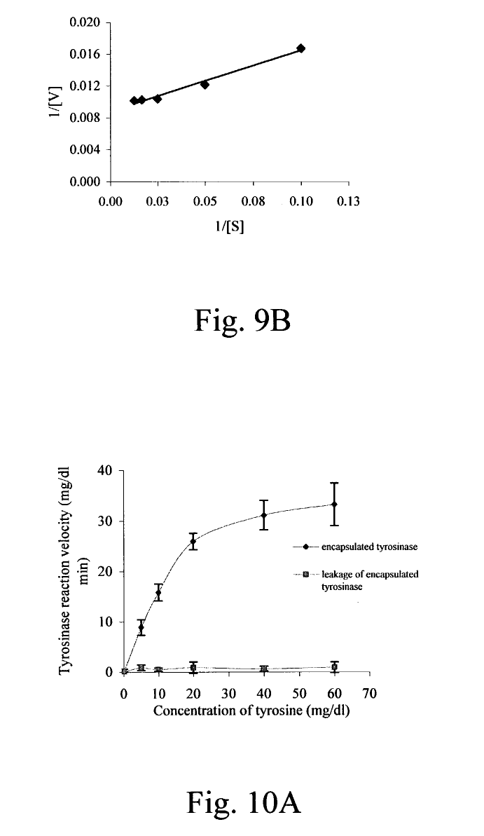

This is to characterize the enzyme kinetics of the preparation and comparing this encapsulated tyrosinse preparation to free tyrosinase. FIG. 9 shows the activity of free tyrosinase in different concentration of tyrosine. The Vmax for 1020 units/3 ml is 114.94 mg/dl-min, and Km is 4.65×104 M.

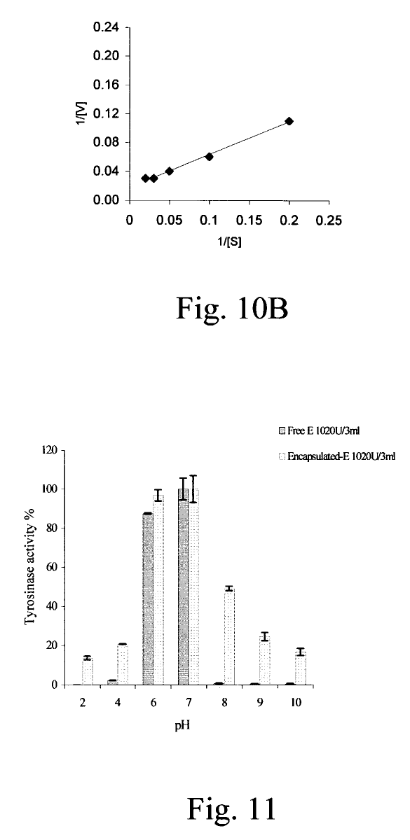

Next, the apparent Vmax and apparent Km value for encapsulated tyrosinase (1020 U/3 ml) were studied. The leakage of encapsulated tyrosinase is also measured. FIG. 10 gives the apparent Vmax is 49.02 mg/dl-min, and its apparent Km is 4.65×10-4 M. From the results obtained, the leakage of encapsulated tyrosinase in the supernatant can be ignored.

From the results obtained (Table 3), the value of apparent Km of micrencapsulated tyrosinase is the same as that of free tyrosinase because the Km value for one enzyme depends on the particular substrate and also environment conditions and ionic. Vmax of the encapsulated tyrosinase is 49.02 mg/dl.min, while that of tyrosinase in free solution is 114.94 mg/dl.min. The higher Vmax value in free tyrosinase than that in encapsulated tyrosinase is due to the inactivation of the enzyme activity during the preparation procedures of microencapsules.

TABLE 3
Summary of studies for Vₘₐₓ and Kₘ in free
and encapsulated tyrosinase

| | Vₘₐₓ (mg/dl · min) | Kₘ (M) |
|---|---|---|
| Free | 114.94 | 4.65 × 10⁻⁴ |
| tyrosinase | | |
| | Apparent Vₘₐₓ | Apparent |
| | (mg/dl · min) | Kₘ (M) |
| Encapsulated | 49.02 | 4.65 × 10⁻⁴ |
| tyrosinase | | |

pH studies in vitro

Orally administered microencapsulated tyrosinase passes through the stomach and intestine having different pH conditions. Thus it is important to analyze the effects of pH. FIG. 11 provides a comparison of free tyrosinase and encapsulated tyrosinase at different pH from 2 to 10. The enzyme activity at pH 7 were taken as 100% original activity, the other data were expressed as percentage of the original activity. At pH 6, encapsulated tyrosinase and free tyrosinase have 97% and 87% of the original activity respectively. At pH 4, encapsulated tyrosinase still has 21% activity, but only 2.3% activity was detected in free tyrosinase. When pH decreases to 2, there is no activity in free enzyme solution, but for encapsulated enzyme, there is still 14% activity remained. Only 0.4-0.6% activity can be tested in free tyrosinase when pH goes up from 8 to 10. For encapsulated tyrosinase, 49% activity remained at pH 8, even at pH 10, 17% activity can be tested. There are two reasons: first, artificial membranes protect enzyme inside, it separates macromolecules, such as proteins, enzyme from outside environment and the hemoglobin is maintained at a high concentration of 10 g/dl inside the microcapsules. Second, high concentration of hemoglobin solution inside artificial cells makes the enzyme more stable than the enzyme in free solution. Furthermore, the hemoglobin solution acts as a buffer, and it can bind hydrogen ions (H+). Thus, hemoglobin solution protects the enzyme inside microencapsules. For free tyrosinase, its activity is easily affected by pH changing.

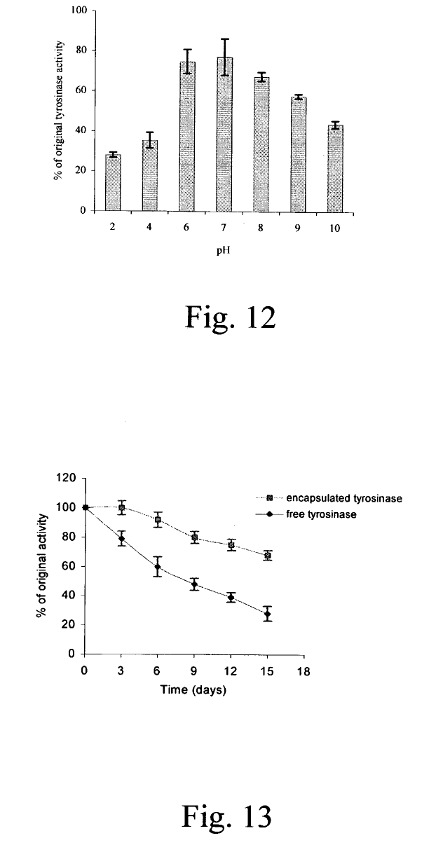

To further simulate the physiological conditions, encapsulated tyrosinase was incubated at 37° C. for one hour at different pH, then washed and tested for enzyme activity at its optimal pH of 7 (FIG. 12). The results showed that at pH 6 to 9, the activity of encapsulated tyrosinase had more than 57% of original activity after one hour incubation at 37° C. FIG. 8 indicates tyrosinase is more sensitive to lower pH, i.e. pH2-4, than other pH range.

### Temperature studies in vitro

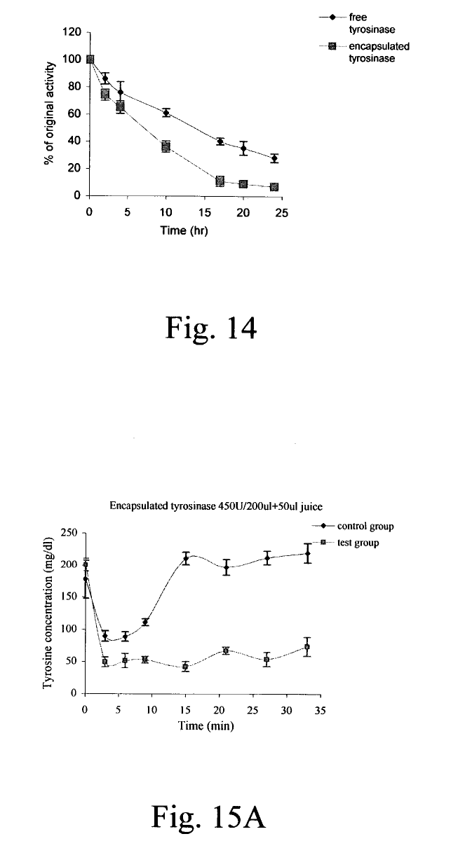

It is important to know the temperature stability of the preparation before carrying out studies in the animal. FIGS. 13 and 14 show the difference for storage stability of free and encapsulated tyrosinase at 4° C. and 37° C. At 4° C. (FIG. 13), encapsulated tyrosinase maintained full activity in the first three days and after 15 days it still had 68% of the original activity. On the other hand, the activity of free tyrosinase went down from the beginning and only had 28% of original activity after 15 days.

At 37° C. (FIG. 14), encapsulated tyrosinase has 61% of original activity after 10 hours and 28% after 24 hours when incubated in water bath at 37° C. This would allow the encapsulated enzyme sufficient stability to carry out its function in the intestine after oral administration. Since the microcapsules stay in the small intestine for about 10-12 hours. For free tyrosinase, it only has 36% of original activity after 10 hours incubation and 7% after 24 hours. The activity of free enzyme decreases faster than that of encapsulated enzyme. Therefore, encapsulated tyrosinase is much more stable than free one under 4° C. and 37° C.

### Incubation with Rat's Intestine Contents in vitro

This is to see the ability of the microencapsulated tyrosinase to lower tyrosinase in intestinal juice before using this for oral administration. Take 50 μl fresh intestine juice from anesthetized rat, then incubate with encapsulated tyrosinase at 37° C. in a shaker. Keep it shake gently in order to make tyrosinase microcapsules react with rat intestine juice completely. In the control group, same amount of microcapsules without enzyme were incubated with rat's intestine juice. Take sample at different time intervals, add 10% trichloroacetic acid (TCA) to stop the reaction, centrifuge it, then analyze the concentration of tyrosine in rat's intestine juice by fluorometric method. In this study, when the activity of encapsulated tyrosinase was increased, and lowering tyrosine level in rat's intestine contents were observed.

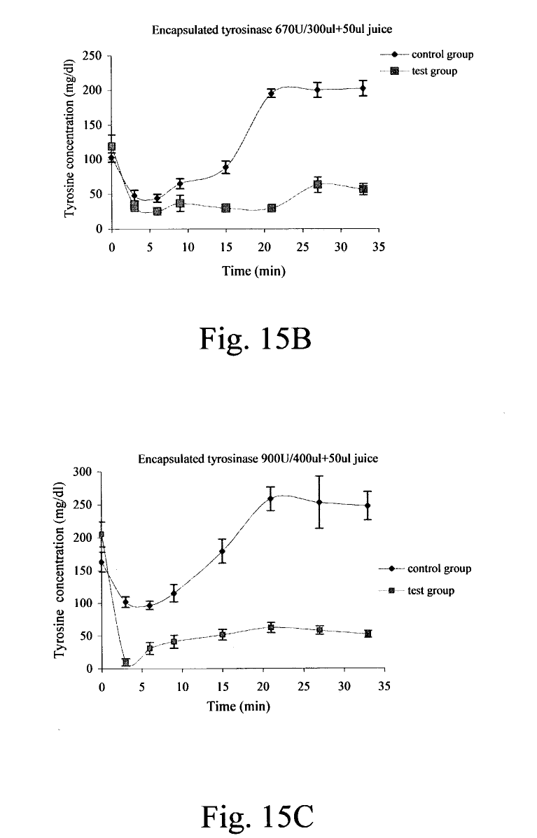

From the results obtained (FIG. 15), a significant different between control group and test group was observed. For the test group, tyrosine concentration in rat's intestine was from 200.25±10.16 mg/dl at the beginning decreased to 73.34±14.72 mg/dl in 30 minutes. Tyrosine level was decreased quickly when incubated with encapsulated tyrosinase for test group. The secretions of intestine contain high concentrations of proteins, enzymes, polypeptides, and peptides. Tryptic enzymes in the intestine break these down into amino acids. Therefore, tyrosine level in control group kept going up with time from 177.58±29.92 mg/dl at the beginning to 219.76±15.21 mg/dl at 30 minutes after an initial decrease due to equilibrate of tyrosine in the intestine juice and the microcapsules. When increasing the volume of encapsulated tyrosinase, tyrosine concentration in test group kept at low level during the experiment period.

### Animal Studies for Oral Administration

Fasted male Sprague-Dawley rats (130-150 g) were used in this studied. All rats were kept in a controlled 12 h light/dark environment with food and water ad libitum. Two groups were studied: (1) control group: feed with artificial cells without enzyme; (2) test group: feed with artificial cells loaded with tyrosinase. Each experiment began on day 0 with blood taken, and no artificial cells were administered on that day. From that day on, and every subsequent day for 21 days, artificial cells were administered orally at 10:00 am, 2:00 pm, and 6:00 pm. Blood samples were taken on Day 4, 8, 11, 15, 18, 22 just after second feeding. Plasma in each blood sample was separated from the blood and placed in 1.5 ml microtube, then stored at −80° C. until analyzed.

### Oral Administration Study in Rats

One-dose a day was not effective in lowering the tyrosine level. Two doses a day and three doses a day were experiment as follows:

### Two-dose Experiment

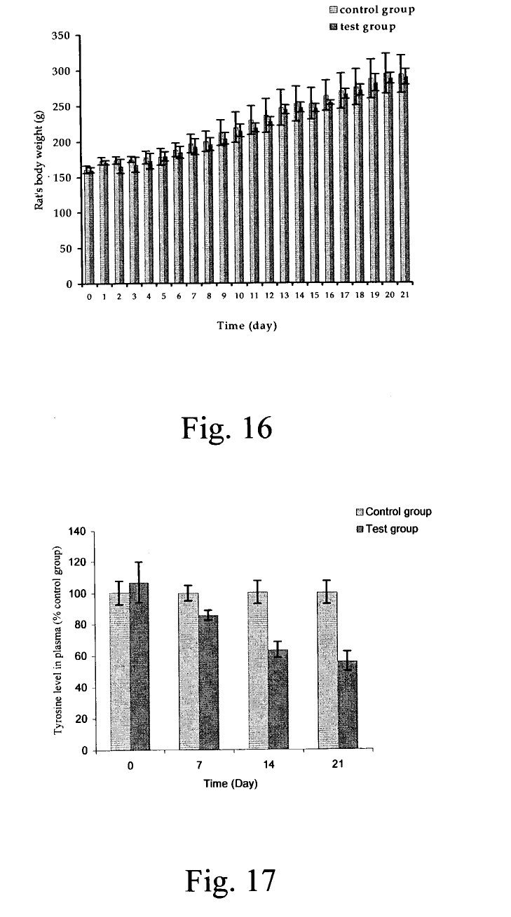

As one dose oral administration of artificial cells could not bring any significant change in the tyrosine level in plasma, dosage was increased to two doses every day. Rats fed on regular rat food and administrated to artificial cells twice a day gained body weight with time during the 21-day experiment period (FIG. 16).

In this study, there was significant difference in tyrosine level in test group starting from the first week and no significant change in control group (FIG. 17). Plotting the results as percent of control group, at Day 7 the tyrosine concentration in the test group is decreased to 85% of that in control group. At Day 14 and Day 21, tyrosine concentration is further decreased in test group to 62.9% and 55.8% respectively. Results showed that encapsulated tyrosinase is effective to lower tyrosine level in the body and longer time is needed to reach this goal. The reason is that once the tyrosine concentration is decreased in plasma, tyrosine inside living cells will come out to compensate the loss of tyrosine in plasma. The basic theory for these experiments is that artificial cells loaded tyrosinase can remove tyrosine from amino acid pool in the intestinal tract and prevent its reabsorb back into the body amino acids pool, thereby lowering circulatory tyrosine level.

Table 4 shows the summary of statistical analysis for tyrosine concentration in two groups. After one week oral administration, there is significant decrease (p<0.005) in tyrosine level in test group compared to that in control group. Continuing this treatment until day 21, the significant decreasing (p<0.0005) in tyrosine level is even greater.

TABLE 4
Compare p-value between Control Group &
Test Group C: control group, T: test group

| Comparing | −value |
|---|---|
| C day 0/ | NS |
| T day 0 | |
| C day 7/ | 0.005 |
| T day 7 | |
| C day 14/ | 0.005 |
| T day 14 | |
| C day 21/ | 0.0005 |
| T day 21 | |

Three-dose Experiment

To decrease tyrosine concentration to a certain level, rats were fed with three oral doses of artificial cells per day. Results showed that the rats in control group and test group continued to get weight during the experiment period (FIG. 18). No abnormal effect or behavior was observed in both groups. From blood sample analysis, there was significant change in tyrosine concentration on day 4 between control group and test group (FIG. 19). The tyrosine concentration in plasma showed constant fluctuations even though the time of feeding and plasma collection was constant. This fluctuation was also found in all experiments, as well as in normal rats. Thus, tyrosine level was taken in control group as 100% original activity, the other data are expressed as percentage of the original activity. On day 4 in test group tyrosine level decreased to 68.8%. On day 18 and day 22, the tyrosine concentration decreased to 56.8% and 52.6% respectively. Results show that 3 doses per day of oral administration can lower tyrosine concentration in rat's plasma from day 4. Table 5 shows the studies of statistical analysis for tyrosine concentration in two groups. At day 4, a significant decrease (P<0.05) in the test group was observed.

TABLE 5
Compare p-value between control group & test group

| Time (Day) | Comparing | P-value |
|---|---|---|
| Day 0 | Control group/Test group | NS |
| Day 4 | Control group/Test group | <0.05 |
| Day 8 | Control group/Test group | <0.05 |
| Day 11 | Control group/Test group | <0.005 |
| Day 15 | Control group/Test group | <0.005 |
| Day 18 | Control group/Test group | <0.005 |
| Day 22 | Control group/Test group | <0.0005 |

NS, not statistically significant (P > 0.05)

### Combine Intravenous Polyhemoglobin and Oral Encapsulated Tyrosinase in Rats

### Combined Animal Studies with Oral Injection and Intravenous Injection

On day 0, take blood sample at 4:00 pm. No artificial cells were administered on this day. From that day on, and every subsequent day for 4 days, artificial cells were administered orally at 10:00 am, 2:00 pm and 6:00 pm. Take blood samples every day just after the second feeding. On day 1, injected 1 ml of PolyHb sample per 250 g rat's body weight to control group and 1 ml of PolyHb-tyrosinase sample per 250 g rat's body weight to test group. On day 2, injected half volume of PolyHb and PolyHb-Tyrosinase sample to rats respectively.

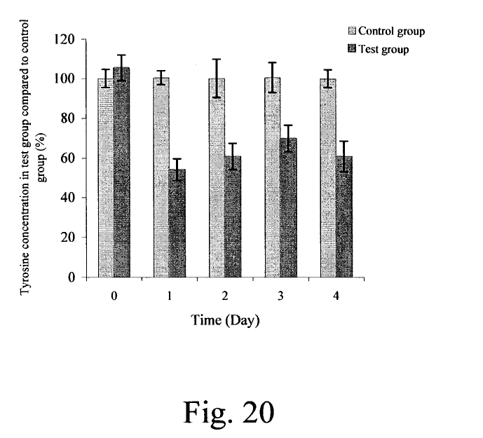

Although intravenous (I.V.) injection provides fast decreasing in tyrosine level in rat's blood system, repeated injection is needed to keep low tyrosine concentration. Crosslinking of the enzyme to polyhemoglobin protects the enzyme from causing immunological reactions. However, long term repeated injections is not as convenient and may cause some reactions. To solve this problem, the two methods were combined. Thus intravenous injection of polyhemoglobin-tyrosinase and Oral administration of artificial cells starts from the beginning of the experiment but only oral administration was continued throughout the experiment period. On day 1, give to the rats the first injection. To keep tyrosine in a low level after the first injection, we further investigate if the rats can stand one more dosage on the second day and whether this second injection will be effective to keep or lower tyrosine level. The body weight of rat in this experiment kept at the same level as that on day 0 except that a slightly decrease on day 2 due to the surgery. On day 1, tyrosine level decreased to 54% after the first injection. Then, on day 2, another half volume of injection was given. This time tyrosine level went up slightly to 61%. On day 3, when stop the injection, tyrosine level went up to 70%, which is 9% higher than the level of yesterday. However, on Day 4, tyrosine level started to decrease which indicated the effectiveness of oral administration of encapsulated tyrosinase (FIG. 20). Results show that I.V. injection method rapidly lowered tyrosine level. Then, oral administration of artificial cells containing tyrosinase keeps that low tyrosine level in the body system. Therefore, combined method can lower tyrosine level in a fast and effective way. Table 6 indicates the statistical studies for tyrosine concentration in both control group and test group.

For control group: feed with artificial cells without enzyme orally three times a day, inject 1 ml per 250 g body weight of PolyHb sample on day 1 and inject half volume of the same sample on Day 2.

For test group: feed with artificial cells loading with tyrosinase orally three times a day, inject 1 ml per 250 g body weight of PolyHb-tyrosinase sample on day 1 and inject half volume of the same sample on day 2.

TABLE 6
Compare p-value between control group & test group

| Time (Day) | Comparing | −value |
|---|---|---|
| Day 0 | Control group/Test | S |
| group | | |
| Day 1 | Control group/Test | 0.0005 |
| group | | |
| Day 2 | Control group/Test | 0.005 |
| group | | |
| Day 4 | Control group/Test | 0.005 |
| group | | |
| Day 5 | Control group/Test | 0.005 |
| group | | |

NS, not statistically significant (P > 0.05)

### Discussion

In this study, PolyHb-tyrosinase and encapsulated tyrosinase were used to decrease tyrosine level in rat's blood system. Furthermore, since crosslinked hemoglobin is oxygen carrier, it provides additional oxygen supply needed for more effective radiation treatment in melanoma which, like other tumours, is not well perfused by oxygen supplying red blood cells. This is in addition to its ability to quickly lower the tyrosine level from the plasma. However, at 24 h after the intravenous injection, tyrosine level went back to the level before injection. Repeated injections are needed to keep tyrosine at low level, and long term repeated daily intravenous injection is not convenience and may also cause reaction. Encapsulated tyrosinase is effective in lowering tyrosine level through the intestinal amino acids pool. However, this method needs longer time to decrease tyrosine level from the body system. In combining these two methods. I.V. injection rapidly lowered the tyrosine level in the plasma, meanwhile oral administration of encapsulated tyrosinase artificial cells kept tyrosine at that low level. Finally, it is assessed the effect of polyhemoglobin-tyrosinase on the growth of melanoma cells in vitro. Result showed that polyhemoglobin-tyrosinase inhibits the growth of melanoma cells which shows that polyhemoglobin-tyrosinase is effective on melanoma cells.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A composition for inhibiting tumor growth, which comprises a tumor growth inhibiting enzymatic moiety in association with at least one protective carrier.

2. The composition of claim 1, wherein said carrier is an oral encapsulation carrier to protect encapsulated enzymatic moiety from digestive enzyme degradation.

3. The composition of claim 2, wherein said encapsulation carrier is a nanocapsule.

4. The composition of claim 1, wherein said enzymatic moiety is selected from the group consisting of tyrosinase, asparaginase and glutaminase

5. The composition of claim 1, further comprising an oxygen binding molecule.

6. The composition of claim 1, wherein said carrier is a molecule biologically active and protective that covalently bonds to said enzymatic moiety.

7. The composition of claim 6, wherein said molecule increase half-life of said enzymatic moiety.

8. The composition of claim 6, wherein said molecule is an oxygen carrying molecule.

9. The composition of claim 8, wherein said molecule is hemoglobin or synthetic hemoglobin.

10. The composition of claim 6, wherein said molecule is albumin or antineoplastic molecule.

11. The composition of claim 10, wherein said antineoplastic molecule is selected from the group consisting of interleukin, interferon α, interferon β, and interferon γ.

12. A method for inhibiting tumor growth in a patient, said method comprising the step of administering an effective amount of the composition of claim 1 to said patient.

13. The method of claim 12, wherein said enzymatic moiety is a tyrosine-degrading and said tumor is a skin cancer tumor.

14. The method of claim 13, wherein said enzymatic moiety is tyrosinase.

15. The method of claim 13, wherein said skin cancer is melanoma.

16. The method of claim 12, wherein said enzymatic moiety is selected from the group consisting of asparaginase and glutaminase.

17. The method of claim 12, wherein said tumor is an amino acid-dependent tumor.

18. The method of claim 12, wherein said tumor is selected from the group consisting of leukemia and lymphoma.
